(19) United States
(12) Patent Application Publication
(10) Pub. No.: US 2020/0381118 A1
(43) Pub. Date: Dec. 3, 2020

(54) ARTIFICIAL INTELLIGENCE (AI) SYSTEM FOR INDIVIDUAL HEALTH MAINTENANCE RECOMMENDATIONS

(71) Applicants: Jana Angelika Hapfelmeier, Sauerlach (DE); Jens Hartel, Kleinmachnow (DE)

(72) Inventors: Jana Angelika Hapfelmeier, Sauerlach (DE); Jens Hartel, Kleinmachnow (DE)

(21) Appl. No.: 16/884,730

(22) Filed: May 27, 2020

Related U.S. Application Data
(60) Provisional application No. 62/855,191, filed on May 31, 2019.

(51) Int. Cl. G16H 50/20; G16H 50/30; G16H 10/60; G16H 10/20

(52) U.S. Cl. CPC G16H 50/20; G16H 10/20; G16H 10/60; G16H 50/30

(57) ABSTRACT

An Artificial Intelligence (AI) system for individual health maintenance recommendations is designed for health maintenance programs. It includes collecting healthcare data from an individual through diagnostic testing, physical examination and questionnaires as well as the individuals personal health goals. The collected data is provided to an AI system having a health database of primary health indicators and environmental and intervention factors to identify the individual's health status, and his health progression and recommendations for interventions and goals to maintain the user's health and to affect socioeconomic outcome measures.

This application claims the benefit of U.S. Provisional Application No. 62/855,191 filed May 31, 2019.

### FIELD OF THE INVENTION

The present invention relates generally to a method and system for providing a comprehensive integrated health maintenance system in order to help users to manage their health risks in a beneficial way. An artificial intelligence (AI) learning system monitors feedback loops from a patient for the constant improvement and personalization of the patient's health maintenance recommendations. The system is part of a service that can be applied in any field of medical help (health maintenance, post treatment care, standard care). However, the flow of all forms of the application are the same. Users that want to take part in the system undergo a medical checkup that shows their health status. Moreover, they present their personal constraints and motivation for the health checkup by a questionnaire. An AI interface predicts how the health and outcome will develop in the future but the goal is a structured and personalized approach for the health maintenance. Moreover, the health goals influence the economic and social burden that come with the health challenges. The system that is presented brings together all components that are necessary for a health recommendation system.

### BACKGROUND OF THE INVENTION

Usually health care is only given to specific diseases and mostly focused on one disease only while care is generally provided when a disease is already present rather than for health maintenance. Medical research has proven that diseases influence each other and that they should be treated in common. The same is also true for health maintenance. Several aspects should therefore be treated together rather than in isolation. Moreover, the patient's preferences and life circumstances should be taken into account to find ways and methods to keep him motivated and produce a healthy outcome. The present health care systems still do not provide a system for health maintenance. Although, there are checkups, which may eventually take place in a physician's office or at work, the results of these checkups are not followed up regularly unless serious diseases are apparent. This lack in health care should be replaced in order to prevent diseases and chronification of diseases which can be treated with a healthier life style. Usually, such a change cannot be done alone but needs the assistance of a coach or another health care professional. They guide a person for a significant time span with individual health recommendations, which are defined based on the person's health profile and the person's personal preferences. Unfortunately, there are not enough people to assist everybody who needs help in their health management. Therefore, a system that is able to automatically extract health care profiles and finds appropriate recommendations is needed. The basis for such a system is health data that is provided by the person himself or by a primary diagnosing unit. Together with individual health goals, the presented system can guide a person to better health. Artificial intelligence (AI) is a branch of computer science which uses computer software to analyze its environment using predetermined rules and search algorithms or pattern recognizing machine learning models to make decisions based on those analyses. An AI system acts with varying degrees of autonomy to reduce manual human intervention for a wide range of functions.

One prior U.S. Patent Application Publication to Ellan et al., (US 2020/0035361) is for a method and electronic device for artificial intelligence based assistive health sensing in an internet of things network using a plurality of electronic devices connected to each other. The system uses health monitoring devices in an A!system for making an appropriate transfer to a health care specialist. After the referral the system stops and the patient may start another monitoring cycle.

### SUMMARY OF THE INVENTION

The present invention is for an AI system for making individual health maintenance recommendations which starts with the collecting of health care data from an individual through diagnostic testing, physical examination and questionnaires and identifying the individual's personal health goals. A health database of primary health indicators and environmental and intervention factors is selected which health database has sources including an incremental feature vector grouping system and a system to evaluate the significance of interventions. A learning artificial intelligence computer system is selected having the selected health database coupled thereto. Data is fed from the collected health care data and the identified personal health goals into the selected artificial intelligence system which generates a personalized intervention recommendation with the artificial intelligence system using a learning graphical system consisting of states and transitions and annotations with the feature vectors of health profiles and health interventions from the health database. The AI system for individual health maintenance produces recommendations for improving the individual's health.

The present invention, more specifically, is for an AI system for individual health maintenance recommendations which is incorporated into a health maintenance service. This starts with the enrollment of a person (also: user of the system). The use is first examined by a doctor, fills out questionnaires about his personal situation and also does diagnostic testing. This data is then provided to the AI system to calculate a personalized health maintenance program for the user. The AI system relies on several external data sources about health development and intervention tracking to calculate health challenges, forecast costs and to extract recommendations for health maintenance. These recommendations are based on the interventions that helped other users that were similar to him and are personalized on the users life circumstances. This includes the person's personal taste, physical activity preferences, familiar and work situation, personal history and experiences. After the recommendation calculation, the users enter the health maintenance program by using three different kinds of channels for health assistance: personal coach, automated recommendation via mobile devices or pure off-line information sharing. This process includes feedback loops where the users give feedback about success and fails to the AI system and recommendations are adopted accordingly. The health maintenance service is finished if specific goals are achieved and the health status is in a steady state. This is followed by a follow up data acquisition that again does diagnostic testing and questionnaires. This evaluation gives hard evidence of the health changes that occurred in the health maintenance service. However, the process also includes a transferal of the user to a specialist or even to an inpatient treatment if the initial diagnostic testing results in major issues that indicate a significant health risk.

### BRIEF DESCRIPTION OF THE DRAWINGS

The accompanying drawings, which are included to provide further understanding of the invention, are incorporated in and constitute a part of the specification and illustrate an embodiment of the invention and together with the description serve to explain the principles of the invention.

In the drawings:

FIG. 1 is a flow diagram of the health challenge retrieval and the specifications for the personalized health goals;

FIG. 2 is an AI model of states of transitions and shows the underlying model that is used to identify health challenges and to give recommendations and measurements for a healthful economic outcome; and

FIG. 3 shows a graphical model consisting of states and transitions reflecting the overall health status of a user of the present system.

### DETAILED DESCRIPTION OF THE PREFERRED EMBODIMENT

The presented invention is directed towards health maintenance services that are based on health data. The artificial intelligence (AI) system for individual health maintenance recommendations (AISHR) is designed to allow a health care system that has a need to keep the population healthy. This is the case for three parties namely: hospitals, company employers and health insurance. They all already have specific programs to keep people healthy for business health management, disease management programs and health support systems but not in an integrated and assisting way. The present system is specially designed for these parties as they are able to collect health data, questionnaires and have direct access to healthcare professionals that can interpret results. Moreover, these instances have the capability to securely send health information to a computer with AI software.

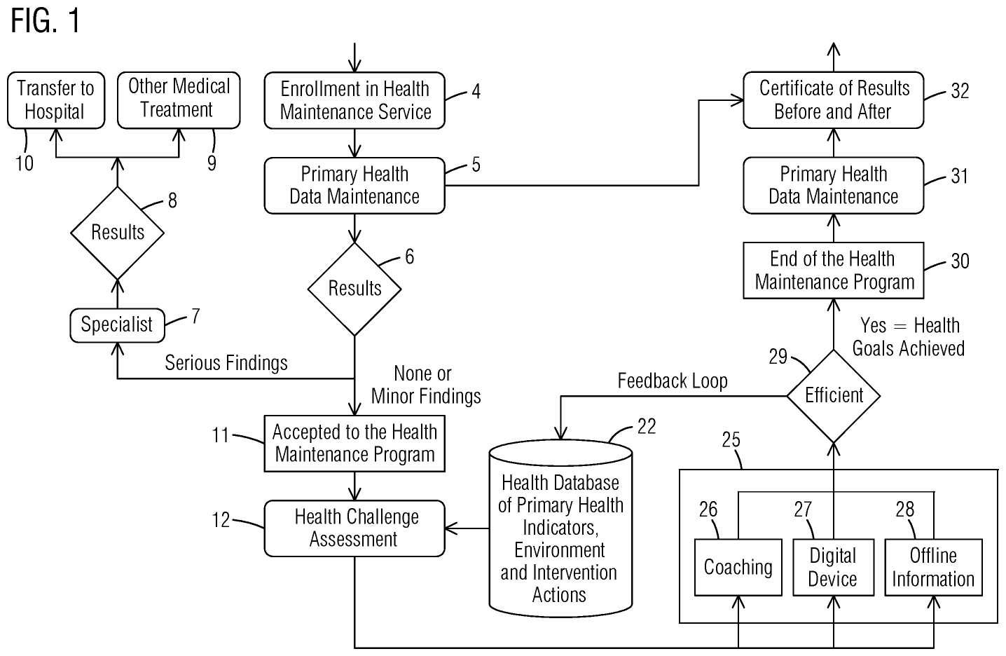

The concept AISHR is illustrated in the flow diagram of FIG. 1. The process is coupled to a health maintenance program in the health care system. The process starts when a user signs into the program (4). Within the program a specific checkup (5) is provided. This can either be done with systems that are already in the health care player's building or they can be provided by a third party. The checkup's goals are to identify specific health parameters that show the vitality of the body system. This includes a blood analysis, cardio and pulmonary evaluation, metabolic analysis, body measurement, sensomotoric tests and a questionnaire about the patient's daily circumstances. The results (6) will show that either the patient is healthy enough to follow the health maintenance process or if there are serious medical problems, is instantly transferred (7) to a specialist. Here again, either the specialist is able to treat the patient himself or, based on his results (8), further transfers (10) him to a hospital where acute major health problems are handled or referred to an appropriate health care provider (9). In the case where the diagnosing reveals an acceptable result, the user is accepted to the AISHR service (11). The next main step is the health challenge assessment (12). There, first the health profile of the user is evaluated and based on that, the user has to provide a health goal that he wants to achieve during the process. This goal can be very individual. Usually, the user has an assistant for this task. This may be a coach, a doctor or another health care related profession.

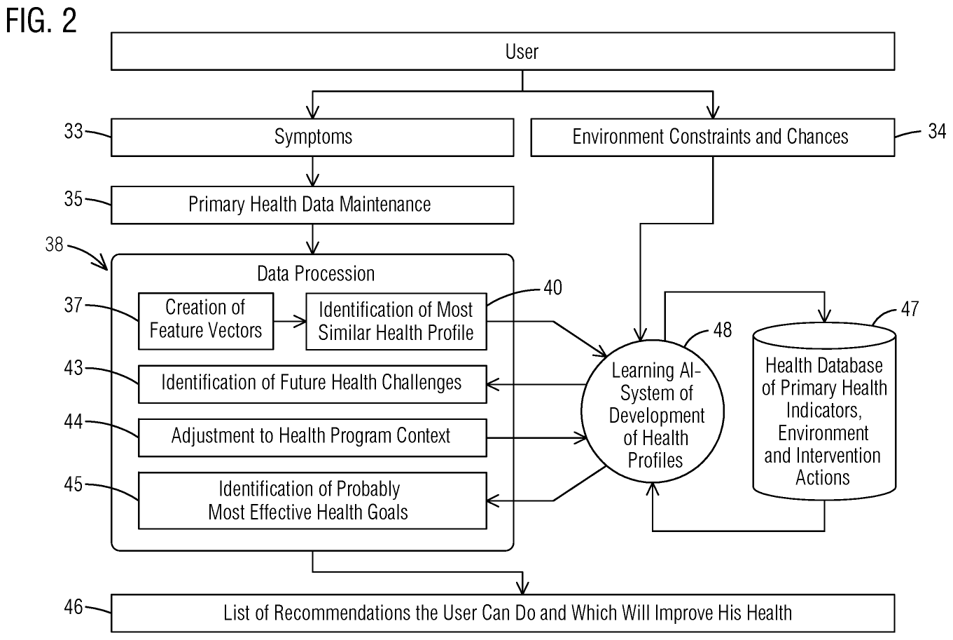

FIG. 2 illustrates this process in detail. The user gives data about his symptoms (33) and constraints (34) in his life as described before. In addition to the primary health data (36) that was taken in the initial assessment, the data procession can start. First, feature vectors are created (37) with the provided data. Therefore, specific health values of the assessment are combined to reflect the overall health status of the user. This feature vector is transferred (40) to identify the most similar health care profile in the AI system (47). The AI system then-gives back the health challenges (43) along with the forecast/prediction of individual health outcomes for this specific user. Then, these forecasts are rated by their health related severity and adjusted to the context (44) in which the person uses the service. This may be, in one case, a decrease of days where he is incapable to work or in another scenario, the medication costs. Then health goals can be set (45) along with the recommendation of interventions that the user should do to achieve that goals (46). For this process to work, a database, which is part of the AI system, is the backend (47) of the service. It captures different data sources and enables the AI system to learn (48) and to improve itself constantly. More specifically, the data from each user's assessment as well as the intervention's success are kept as well as additional information from the environment that may influence the success of the intervention program, e.g. whether data or local specialties.

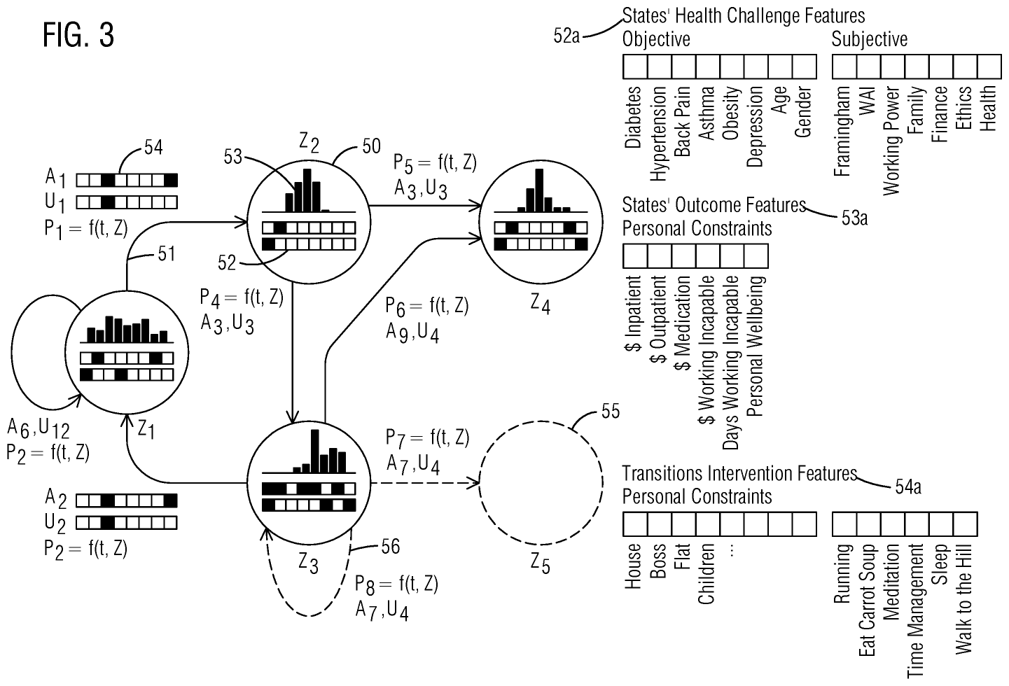

The AI system is specified in more detail in FIG. 3. The AI component is based on a graphical model consisting of states (50) and transitions (51). States reflect the overall health status (or health profile) of users while the transitions indicate which health states may follow each other. The information is learned from all the data that is located in the backend database (47) (of FIG. 2). States are annotated with a profile vector that specifies the typical distribution of objective and subjective health features (52 & 52a). Furthermore, the states protocol important health outcomes like health related costs, well being or incapability to work (53&53a). This information is used to forecast the socioeconomic development of a user. The transitions are also labeled. Here, the information is stored, what the user actually did between states (54 & 54a). By comparing different transitions that origin from the same state, one can evaluate which interventions had a significant influence on the afterwards outcome. Additionally, the transitions keep the time information and give probabilities about the occurrence of the transition. This probability is modeled in dependence of specific time frames, i.e. months or years. This depends on the actual health service. As constant information is arriving in the system, it is able to update itself by adjusting profiles, probabilities or outcome distributions. Additionally, new health states (55) can be automatically created as well as new transitions (56). If evidence indicate that two states do not differ enough, they can be merged to one state.

The main challenge here is to identify the health states. This is done by aggregating similar users, i.e. their health profiles. Then, weighted average vectors are calculated for their profiles as well as their outcomes. This can only be done by using matching algorithms between different data sources, e.g. claims data and primary care data.

Now that health challenge assessment is done as shown in FIG. 3 and the personalized recommendations are extracted, the user is given assistance. This may be a personal coach (26), a digital device (27) or offline information (28). It may also be a combination of these three (25) depending on the user's preferences. All assistant components help the user to achieve his goal. This is again measured by user feedback (29) whether to be efficient, i.e. whether the goals can be achieved with the recommendations that are given. This feedback is again incorporated into the backend database 22 and helps to adjust the recommendations. It may also be the case, that the health status of the user significantly changes and the health challenge assessment is triggered again (12). Then other recommendations may be given. This process continues until the service is considered efficient. Then the health service program ends for the user (30). Afterwards, a primary health data assessment collects data about the objective change in the health status of the patient (31). He receives a certificate that illustrates his achievements during the service (32).

It should be clear at this time that an artificial intelligence system for an individual health maintenance recommendations has been provided. However the present invention is not to be considered limited to the forms shown which are to be considered illustrative rather than restrictive.

We claim:

1. An AI system for making individual health maintenance recommendations comprising the steps of:
collecting health care data from an individual through diagnostic testing, physical examination and questionnaires;
identifying the individual's personal health goals;
selecting a health database of primary health indicators and environmental and intervention factors, said health database having sources including an incremental feature vector grouping system and a system to evaluate significance of interventions;
selecting a learning artificial intelligence system having said health database coupled thereto;
feeding said collected health care data and said identified personal health goals into said selected artificial intelligence system; and
generating a personalized intervention recommendation with said artificial intelligence system using a learning graphical system, consisting of states and transitions and annotations with said feature vectors of health profiles and health interventions from said health database;
whereby an AZ system for individual health maintenance produces recommendations for improving one's health.

2. The AI system for individual health maintenance recommendations in accordance with claim 1 including a feedback loop from said individual for the constant improvement of the user's health maintenance recommendations.
